(19) United States
(12) Patent Application Publication
(10) Pub. No.: US 2022/0087344 A1
(43) Pub. Date: Mar. 24, 2022

(54) A TEXTILE ELEMENT SUCH AS A CLOTHING

(71) Applicant: CLIM8, Tassin-La-Demi-Lune (FR)

(72) Inventors: Julien GUERITEE, Eveux (FR); Florian MIGUET, Venissieux (FR); Pierre MOUETTE, HK (CN)

(73) Assignee: CLIM8, Tassin-La-Demi-Lune (FR)

(21) Appl. No.: 17/423,452

(22) PCT Filed: Jan. 29, 2019
(86) PCT No.: PCT/IB2019/050707

§ 371 Date: Jul. 15, 2021

(51) Int. Cl. A41D 19/015; A41D 1/00; A41D 13/00; H05B 1/02

(52) U.S. Cl. CPC A41D 19/01535; A41D 1/002; H05B 1/0272; A41D 19/01541; A41D 13/0005

(57) ABSTRACT

The invention relates to a textile element, such as a clothing, aimed at covering a part of a body, such as a human body, with a particular application to a clothing for generating a physical effect, such as heat or cold, to the part of the body.

The textile element 1 forms an envelop 1 aimed at covering at least partially a part of a body, said textile element comprising a textile support and body part detection means 2 located on or in said textile support, said body part detection means 2 being configured to detect the presence and/or absence of said part of a body inside the envelop 1 and to generate a detection signal when presence and/or absence of said part of a body inside the envelop 1 is detected.

### FIELD OF THE INVENTION

The present invention relates to a textile element, such as a clothing, aimed at covering a part of a body, such as a human body, with a particular application to a clothing for generating a physical effect, such as heat or cold, to the part of the body.

### BACKGROUND OF THE INVENTION

Thermal comfort is driven by the temperature inside a person's body and the temperature at the surface of your body. Deep body temperature of a person is generally 37° C. and the mean skin temperature of a person is generally 33° C. People become uncomfortable in a thermal sense when the environment changes for example, increased wind or extra sunny day, or when a person moves to a cooler place. In common situations, discomfort in a thermal sense is felt by a user when the user's skin temperature changes, rather than a change in core body temperature.

Conventional chemical or electric heating systems used in clothing can easily deliver heat at relatively high levels. Most currently available devices consist of a wearable garment with heating pads that can be manually adjusted by the user. In at least some devices the heating pads produce an equal heat output and all the heating pads are activated to provide heat.

Let us consider the example of a warming glove with a heating element activated when a sensed temperature falls below a determined threshold. If the glove is connected to its external power source, and the conditions for triggering the heating are met, the heating element will be activated even if it is not worn by a user. For example, when the glove is stored in a room which is not heated, and still connected to its external power source because the user has forgotten to disconnect it, the heating element could be automatically activated, depending on the ambient temperature, which leads to unloading of the power source.

This problem could be solved by integrating to the glove a manual on/off button. Still, the user might forget to switch the glove off. Besides, the user has to think of switching the glove on again the next time he/she wears the glove. On top of this, integration of such a button in the glove has ergonomic consequences for the user.

In other applications, some other actions should be authorized only when the hand of the user is inserted into the glove, for example for safety reason. This is the case with some dangerous tools, which should not be used by a user if the latter does not wear protective gloves.

### SUMMARY OF THE INVENTION

The invention aims therefore at solving the problems mentioned, amongst other problems.

To this end, the invention proposes a textile element forming an envelop aimed at covering at least partially a part of a body, which is able to determine when the part of a body is inserted into the envelop.

According to a first aspect, the object of the invention is a textile element forming an envelop aimed at covering at least partially a part of a body, said textile element comprising a textile support and body part detection means located on or in said textile support, said body part detection means being configured to detect the presence and/or absence of said part of a body inside the envelop and to generate a detection signal when presence and/or absence of said part of a body inside the envelop is detected.

In some embodiments, the textile element further comprises one or more of the following features, considered alone or according to any technically possible combination:
the textile element comprises an active element adapted for generating a physical effect, such as heat or cold, and located on or in the textile support, the active element being adapted for being connected to a power source and a control unit configured to allow the activation/deactivation of said active element, the body part detection means being adapted for being connected, and for transmitting the detection signal, to said control unit, such as to allow the control unit to activate/deactivate the active element when receiving said detection signal;
the body part detection means are configured to detect the part of a body entering inside, and/or going out of, the envelop;
the body part detection means comprise sensing means adapted for sensing a physical information related to the presence and/or the absence of the part of a body inside the envelop, and a controller connected to the sensing means and configured to receive one or more sensed information from the sensing means and to generate the detection signal on the basis of the one or more sensed information;
the information sensed by the sensing means is related to the contact of the hand with the sensing means or to the hand coming in a determined vicinity of the sensing means;
the body part detection means comprise capacitive detection means, the sensing means comprising capacitive sensing elements, the controller being configured to detect an electric field generated when the part of a body comes in contact with, or in a vicinity of said capacitive sensing element;
the capacitive sensing elements comprise one or more electrically conductive elements located in or on the textile support, the controller being configured to detect an electric field generated when the part of a body comes in contact with, or in a vicinity of, at least one of said conductive elements;
the sensing means are disposed on or in the textile support such as to form one or more sensing paths running through one or more predetermined points of the textile element and aimed at being in contact with, or in the vicinity of, corresponding predetermined points of the part of a body;
the sensing means form at least two distinct sensing paths;
the part of the body is a hand and the textile element is a glove, at least one of the sensing paths running such as to go from the back part of the glove, between the thumb and index finger parts of the glove, to the palm part of the glove;
the part of the body is a hand and the textile element is a glove, at least one of the sensing paths running on the palm part of the glove such as to go through a line formed by the based points where each of the finger parts, but the thumb part, of the glove joins the palm part of the glove;
the sensing means comprise pressure sensing elements adapted to sense a pressure from the part of a body inside the envelop;
the textile element comprises movement detection means, such as an accelerometer, adapted for being connected to a power source and the control unit, the movement detection means being adapted for being connected, and for transmitting a movement detection signal, to said control unit, such as to allow the control unit to activate/deactivate the active element when receiving said movement detection signal;
the body is a human body, and the textile element is a clothing configured to receive at least a part of said body;
the body is a hand and the clothing is a glove, or the part of the body is a foot and the clothing is a sock or a shoe, or the part of the body is the head and the clothing is a cap or a helmet.

Such a textile element allows therefore the determination of the presence and/or absence of the part of a body inside the envelop. This allows in turn, for example in the application of a heating glove mentioned above, to save energy from the power source by not activating uselessly the heating without requiring a manual intervention from the user. This allows too, in other applications, to prevent a user to make an action when he/she does not wear the glove.

### DRAWINGS

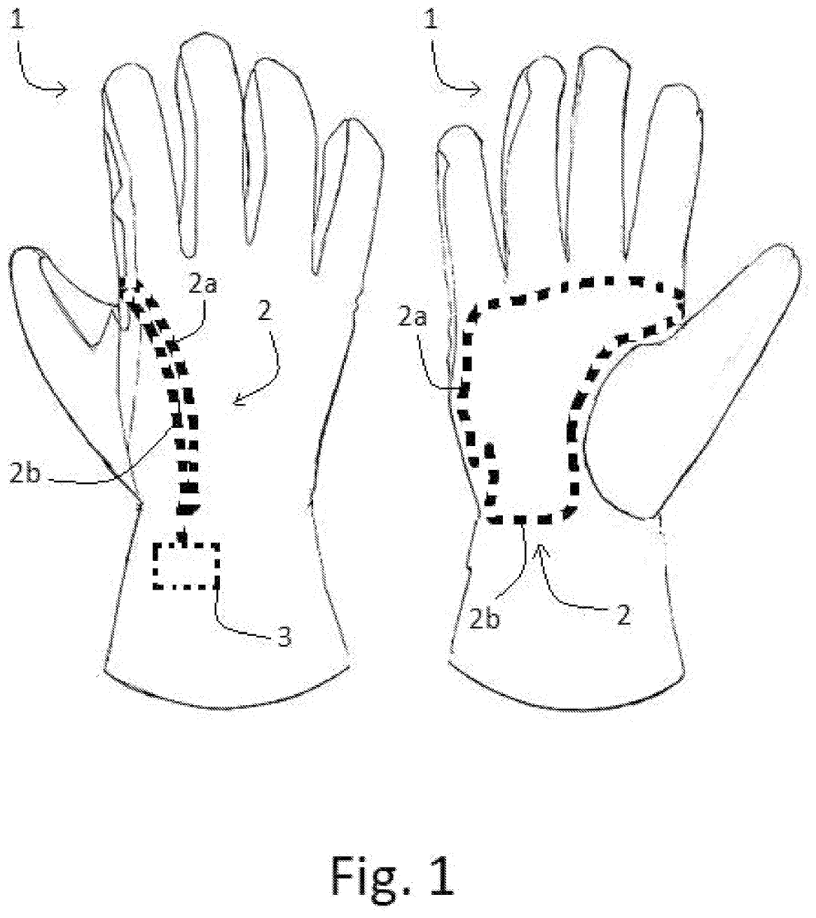

The invention and its advantages may be better understood by referring to the description which follows, given as example and for illustrative purpose only, and by referring to FIG. 1 which shows an example of a textile element according to the invention, namely a glove.

### DETAILED DESCRIPTION

FIG. 1 illustrates an example of application of the invention to the technical field of clothing. More specifically, this example deals with the application where the textile element 1 is a glove, and the part of a body is the hand of a user.

The glove 1 forms an envelop 1 which aims at covering totally or partially the hand of a user.

Detection means 2 are integrated into the glove 1, more specifically into a textile support of this glove 1. This means that the glove 1 can be made of one or more textile layer, one of which being said textile support. Integration of the detection means 2 into the textile support means that these detection means are located on or in the textile support, for example by knitting, stitching and/or thermal bonding.

The hand detection means 2 are configured to detect the presence and/or absence of the hand of a user inside the envelop formed by the glove 1, and to generate a detection signal when such presence and/or absence of the hand inside the envelop 1 is detected.

By presence and/or absence of the hand respectively in and/or from the glove, it may be understood entering and/or retrieving the hand respectively in and/or from the glove. In such a case, the detection means 2 are configured to detect the hand entering inside, and/or going out of, the envelop formed by the glove 1. This means that the detection of presence may take place at one specific moment of the phase of inserting the hand into the glove depending on calibration and presetting, not necessarily when the hand is fully inserted into the glove. Similarly, the detection of absence may take place at one specific moment of the phase of retrieving the hand out of the glove depending on calibration and presetting, not necessarily when the hand is fully retrieved out of the glove.

In a preferred embodiment, the textile element, such as the glove 1 of FIG. 1, is an active textile element. Such an active textile element comprises an active element (not represented on FIG. 1) adapted for generating a physical effect, such as heat or cold. This active element is located on or in the textile support, and is adapted for being connected to a power source and a control unit (both not represented on FIG. 1), the control unit being configured to allow the activation/deactivation of said active element.

Such an example of configuration of a textile element with an active element adapted for generating heat when powered by a power source and under control of a control unit, is described in WO2018214088 or WO2017162131.

The body part detection means 2 are then adapted for being connected, and for transmitting the detection signal, to said control unit, such as to allow the control unit to activate/deactivate the active element when receiving said detection signal.

Therefore, in the specific embodiment of a heating glove, the detection of the hand inserted into, or retrieved from, the glove, allows to automatically activate, or deactivate, the heating element, through the controlling unit which receives the detection signal from the detection means.

Whether in the embodiment of a heating glove or in other embodiments, the hand detection means 2 more precisely comprise sensing means 2a, 2b and a controller 3.

The sensing means 2a, 2b are adapted for sensing physical information related to the presence and/or the absence of the hand inside the envelop 1 by the glove.

The controller 3 is connected to the sensing means 2a, 2b. and configured to receive one or more sensed information from the sensing means 2a, 2b and to generate the detection signal on the basis of the one or more sensed information.

The information sensed by the sensing means is for example an information related to the contact of the hand with the sensing means 2a, 2b or related to the hand coming in a determined vicinity of the sensing means 2a, 2b

As an example, the detection means 2 comprise capacitive detection means 2, the sensing means 2a, 2b comprising then capacitive sensing elements 2a, 2b. The controller 3 is then configured to detect an electric field generated when the hand comes in contact with, or in a vicinity of, the capacitive sensing elements 2a, 2b.

Alternatively, or in combination, de detection means 2 may comprise pressure sensing means, the sensing means comprising then pressure sensing elements adapted to sense a pressure from the hand inside the glove 1.

In case of capacitive detection means 2, the capacitive sensing means 2a, 2b comprise one or more electrically conductive elements 2a, 2b. These conductive elements 2a, 2b can be conductive wires, such as represented in FIG. 1, or plates in conductive material such as copper, directly located, or located via a support such as a PCB support, in or on the textile support.

The controller 3 is then configured to detect an electric field generated when the hand comes in contact with, or in a vicinity of, at least one of the conductive elements 2a, 2b.

When connected to a power source, through the controller 3, the conductive elements 2a, 2b generate an electric field, the intensity of which being modified when the hand of a user comes in the vicinity of these conductive elements 2a, 2b.

The sensing means 2a, 2b are disposed on or in the textile support such as to form one or more sensing paths 2a, 2b, precisely two distinct sensing paths 2a, 2b in the example of FIG. 1.

One sensing path is enough, but two are preferred in order to reduce the risk of false detection.

The one or more sensing paths 2a, 2b run through one or more predetermined points of the textile element, depending on the application. These sensing paths 2a, 2b aim at being in contact with, or in the vicinity of, corresponding predetermined points of the part of the body, thus the hand in our example.

In the example of a hand and a glove illustrated in FIG. 1, the two sensing paths 2a, 2b run such as to go from the back part of the glove (left part of FIG. 1), between the thumb and index finger parts of the glove, to the palm part of the glove (right part of FIG. 1).

In the case of conductive wires 2a, 2b, one end of each of these wires 2a, 2b is free (right part of FIG. 1), and the other end is connected to the controller 3 (left part of FIG. 1), such as a microchip 3.

As can be seen of the right part of FIG. 1, one of the sensing paths 2a, 2b, namely the sensing path 2a, runs on the palm part of the glove such as to go through a line formed by the based points where each of the finger parts, but the thumb part, of the glove joins the palm part of the glove.

Initially, a threshold value of electric field is determined, from or above which it is considered that the hand is inside the glove 1, the noise being taken into account in this determination.

The conductive elements 2a, 2b have to be positioned such as to avoid crossing any other conductive elements such as data or power wires.

The controller 3 is configured to detect when the electric field generated goes above a first predetermined value (presence) and below second predetermined value (absence), the first and second predetermined values being potentially identical. The derivative, and possibly the integral, of the electric field generated over one or more predetermined periods, may also be used to render the detection even more precise.

Various methods may be used to generate the detection signal on the basis of the electric field generated.

In a specific embodiment, the controller 3 sends one or more pulses to these capacitive conductive elements 2a, 2b in a sensing cycle, and measures the pulse responses during this sensing cycle, the latter depending on the electric capacity of the conductive elements 2a, 2b, therefore on the electric field generated. The pulse responses over one cycle are accumulated in a counter, the latter being reinitialized each beginning of cycle. A based value, corresponding to the absence of the hand inside the glove, is stored in the memory of the controller 3. At the end of each sensing cycle, the value accumulated in the counter is compared to the based value, and the positive or negative delta between the accumulated value and the base value is used to determine if the hand has been inserted or not into the glove.

With capacitive detection means 2, in some situations, for example in case of high humidity rate, the electric field detection can be disturbed, with increase of risk of false detection. In such a case, it can be advantageous to use a movement detection means also integrated into the glove, such as an accelerometer.

This movement detection means is adapted for being connected to a power source and the control unit mention previously regarding the embodiment of an active textile element with an active element such as a heating or cooling element.

This movement detection means is also adapted for transmitting a movement detection signal to the control unit, such as to allow the control unit to activate/deactivate the active element when receiving a movement detection signal.

In such a way, even if the main detection means is disturbed, for example because of a high humidity rate, a movement detected by the movement detection means, after a period of rest, can be interpreted such as to wake up the heating system. Similarly, a lack of movement detected by the movement detection means, after a period of activity, can be interpreted such as to stop the heating system.

The above description has been directed to specific embodiments of this invention which is, however, not limited to these embodiments described for purpose of example only.

In particular, the invention is not limited to a textile element, or clothing, aiming at covering a part of a human body, but extends to a textile element aiming at covering a part of an animal body or any sort of non-living body.

In the case of a part of a human body, the invention is not limited to a glove, but extends to clothing of any sort aiming at covering at least partially said part of a human body, such as a sock or a schoe (the part of a body being then a foot), or a cap or a helmet (the part of a body being then the head).

Besides, the invention is not limited to any specific kind of sensing technology for detecting the presence and/or absence of the body part inside the envelop formed by the textile element. A preferred example is given in the description that is based on capacitive sensing means, but other types of sensing means could be envisaged, such as pressure sensing means, with pressure sensors arranged on one or more sensing path as described above.

1. A textile element forming an envelop aimed at covering at least partially a part of a body, said textile element comprising a textile support and body part detection means located on or in said textile support, said body part detection means being configured to detect presence or absence, or presence and absence, of said part of a body inside the envelop and to generate a detection signal when presence or absence, or presence and absence, of said part of a body inside the envelop is detected.

2. The textile element according to claim 1, wherein it comprises an active element adapted for generating a physical effect, and located on or in the textile support, the active element being adapted for being connected to a power source and a control unit configured to allow the activation/deactivation of said active element, the body part detection means being adapted for being connected, and for transmitting the detection signal, to said control unit, such as to allow the control unit to activate/deactivate the active element when receiving said detection signal.

3. The textile element according to claim 1, wherein the body part detection means are configured to detect the part of a body entering inside, or going out of, or entering inside and going out of, the envelop.

4. The textile element according to claim 1, wherein the body part detection means comprise sensing means adapted for sensing a physical information related to the presence or the absence, or the presence and the absence, of the part of a body inside the envelop, and a controller connected to the sensing means and configured to receive one or more sensed information from the sensing means and to generate the detection signal on the basis of the one or more sensed information.

5. The textile element according to claim 4, wherein the information sensed by the sensing means is related to the contact of the hand with the sensing means or to the hand coming in a determined vicinity of the sensing means.

6. The textile element according to claim 4, wherein the body part detection means comprise capacitive detection means, the sensing means comprising capacitive sensing elements, the controller being configured to detect an electric field generated when the part of a body comes in contact with, or in a vicinity of said capacitive sensing elements.

7. The textile element according to claim 6, wherein the capacitive sensing elements comprise one or more electrically conductive elements located in or on the textile support, the controller being configured to detect an electric field generated when the part of a body comes in contact with, or in a vicinity of, at least one of said conductive elements.

8. The textile element according to claim 4, wherein the sensing means comprise pressure sensing elements adapted to sense a pressure from the part of a body inside the envelop.

9. The textile element according to claim 4, wherein the sensing means are disposed on or in the textile support such as to form one or more sensing paths running through one or more predetermined points of the textile element and aimed at being in contact with, or in the vicinity of, corresponding predetermined points of the part of a body.

10. The textile element according to claim 9, wherein the sensing means form at least two distinct sensing paths.

11. The textile element according to claim 8, wherein the part of the body is a hand and the textile element is a glove, at least one of the sensing paths running such as to go from the back part of the glove, between the thumb and index finger parts of the glove, to the palm part of the glove.

12. The textile element according to claim 8, wherein the part of the body is a hand and the textile element is a glove, at least one of the sensing paths running on the palm part of the glove such as to go through a line formed by the based points where each of the finger parts, but the thumb part, of the glove joins the palm part of the glove.

13. The textile element according to claim 2, wherein it comprises movement detection means, adapted for being connected to a power source and the control unit, the movement detection means being further adapted for being connected, and for transmitting a movement detection signal, to said control unit, such as to allow the control unit to activate/deactivate the active element when receiving said movement detection signal.

14. The textile element according to claim 1, the body being a human body, and the textile element being a clothing configured to cover at least partially a part of said body.

15. A clothing according to claim 14, wherein the part of the body is a hand and the clothing is a glove, or the part of the body is a foot and the clothing is a sock or a shoe, or the part of the body is the head and the clothing is a cap or a helmet.

16. The textile element according to claim 2, wherein the physical effect is heat or cold,

17. The textile element according to claim 13, wherein the movement detection means comprise an accelerometer.
